(19) United States
(12) Patent Application Publication
(10) Pub. No.: US 2010/0048968 A1
(43) Pub. Date: Feb. 25, 2010

(54) PROCESS FOR THE CONVERSION OF LOWER ALKANES TO AROMATIC HYDROCARBONS AND ETHYLENE

(76) Inventors: Ann Marie Lauritzen, Houston, TX (US); Ajay Madhav Madgavkar, Katy, TX (US)

Correspondence Address: SHELL OIL COMPANY, P O BOX 2463, HOUSTON, TX, 772522463, US

(21) Appl. No.: 12/542,420

(22) Filed: Aug. 17, 2009

Related U.S. Application Data
(60) Provisional application No. 61/089,930, filed on Aug. 19, 2008.

(51) Int. Cl. C07C 15/04; C07C 15/00

(52) U.S. Cl. 585/413

(57) ABSTRACT

The present invention provides an integrated process for producing ethylene and aromatic hydrocarbons, specifically benzene, which comprises: (a) contacting a mixed lower alkane feed with an aromatic hydrocarbon conversion catalyst to produce a product mixture which is comprised of aromatic reaction products including benzene, unreacted ethane and non-aromatic products, (b) separating and recovering the benzene and any other aromatic reaction products, (c) separating and recovering the ethane, and (d) introducing the ethane into a cracker to produce ethylene.

### CROSSREFERENCE TO RELATED APPLICATION

This application claims priority to U.S. Provisional Application Ser. No. 61/089,930 filed Aug. 19, 2008, the entire disclosure of which is hereby incorporated by reference.

### FIELD OF THE INVENTION

The present invention relates to an integrated process for producing aromatic hydrocarbons and ethylene from lower alkanes. More specifically, the invention relates to an integrated process for the production of benzene and ethylene from lower alkanes with lower capital and operating costs.

### BACKGROUND OF THE INVENTION

Benzene and ethylene are two of the most important basic products of the modern petrochemicals industry. Benzene is used to make key petrochemicals such as styrene, phenol, nylon and polyurethanes, among others. Ethylene is used in the manufacture of other petrochemicals such as polyethylene, ethylene oxide, ethylene dichloride, and ethylbenzene, among others.

Generally, benzene and other aromatic hydrocarbons are obtained by separating a feedstock fraction which is rich in aromatic compounds, such as reformates produced through a catalytic reforming process and pyrolysis gasolines produced through a naphtha cracking process, from non-aromatic hydrocarbons using a solvent extraction process. However, in an effort to meet a projected aromatics supply shortage, numerous catalysts and processes for on-purpose production of aromatics (including benzene) from alkanes containing six or less carbon atoms per molecule have been investigated. The ease of conversion of individual alkanes to aromatics increases with increasing carbon number and thus mixed alkane feeds have been considered. For example, U.S. Pat. No. 5,258,564 describes a process for converting C₂ to C₆ aliphatic hydrocarbons to aromatics comprising contacting the feed with a catalyst at deehydrocyclodimerization conditions wherein the catalyst comprises a zeolite having a Si:Al ratio greater than 10 and a pore diameter of 5-6 Angstroms, a gallium component and an aluminum phosphate binder.

The catalysts used are usually bifunctional, containing a zeolite or molecular sieve material to provide acidity and one or more metals such as Pt, Ga, Zn, Mo, etc. to provide dehydrogenation activity. For example, U.S. Pat. No. 4,350,835 describes a process for converting ethane-containing gaseous feeds to aromatics using a crystalline zeolite catalyst of the ZSM-5-type family containing a minor amount of Ga. As another example, U.S. Pat. No. 7,186,871 describes aromatization of C₁-C₄ alkanes using a catalyst containing Pt and ZSM-5.

Ethylene is generally made from ethane and/or higher hydrocarbons in a high-temperature thermal or catalytic cracker unit. The manufacture of olefins by hydrocarbon cracking is a well-established commercial process which is described in “Ethylene: Keystone to the Petrochemical Industry” by Ludwig Kniel, Marcel Dekker Publisher (1980).

When a feed of ethane plus one or more higher hydrocarbons is converted into olefins in a cracker unit, it results in production of other olefins in addition to ethylene. These include propylene, butylenes, butadiene, pentenes, etc., depending on the composition of the cracker feedstock. The product separation scheme for such a mixed feed cracker tends to be complicated by the presence of multiple olefin products which in many cases have to be separated from other similar molecules (such as the corresponding paraffins) to meet the product specifications. The end result is that the capital expenditure as well as the operating costs of such a cracker complex are much higher than those of a cracker which produces only ethylene from a mainly ethane feedstock.

It would be advantageous to provide a lower alkane dehydroaromatization process wherein (a) lower cost ethylene can be produced as a coproduct and (b) the feed to the dehydroaromatization reactor is substantially converted, thus avoiding any feed recycle and resulting in lower capital and operating costs.

### SUMMARY OF THE INVENTION

The present invention provides an integrated process for producing ethylene and aromatic hydrocarbons, specifically benzene, which comprises:

(a) contacting a mixed lower alkane feed with an aromatic hydrocarbon conversion catalyst to produce a product mixture which is comprised of aromatic reaction products including benzene, unreacted ethane and non-aromatic products,

(b) separating and recovering the benzene and any other aromatic reaction products,

(c) separating and recovering the ethane, and

(d) introducing the ethane into an alkane cracker, preferably a thermal or catalytic cracker, to produce ethylene.

In another embodiment, benzene may be separated from toluene and/or xylene and C₉₊ aromatic products in step (b) and the benzene may be recovered. The toluene and/or xylene may then be hydrodealkylated to produce additional benzene.

### BRIEF DESCRIPTION OF THE DRAWINGS

FIG. 1 is a flow diagram which illustrates the conversion of a mixed lower alkane stream into aromatics and ethane which is then cracked to produce ethylene.

FIG. 2 is a flow diagram which illustrates the conversion of a mixed lower alkane stream into aromatics and ethane which is then cracked to produce ethylene and wherein benzene is separated from toluene and xylene which are hydrodealkylated to produce more benzene.

### DETAILED DESCRIPTION OF THE INVENTION

This invention relates to an integrated processing scheme for producing benzene (and other aromatics) and ethylene from a mixed lower alkane stream which may contain C₂, C₃, C₄ and/or C₅ alkanes (referred to herein as “mixed lower alkanes” or “lower alkanes”), for example an ethane/propane/butane-rich stream derived from natural gas, refinery or petrochemical streams including waste streams. Examples of potentially suitable feed streams include (but are not limited to) residual ethane and propane from natural gas (methane) purification, pure ethane, propane and butane streams (also known as Natural Gas Liquids) co-produced at a liquefied natural gas site, C₂-C₅ streams from associated gases co-produced with crude oil production, unreacted ethane “waste” streams from steam crackers, and the C₁-C₃ byproduct stream from naphtha reformers. The lower alkane feed may be deliberately diluted with relatively inert gases such as nitrogen and/or with various light hydrocarbons and/or with low levels of additives needed to improve catalyst performance. The primary desired products of the process of this invention are benzene, toluene, xylene and ethylene.

The hydrocarbons in the feedstock may include ethane, propane, butane, and/or C₅ alkanes or any combination thereof. Preferably, the majority of the mixed alkanes in the feedstock is ethane and propane. The feedstock may contain in addition other open chain hydrocarbons containing between 3 and 8 carbon atoms as coreactants. Specific examples of such additional coreactants are propylene, isobutane, n-butenes and isobutene. The hydrocarbon feedstock preferably is comprised of at least about 30 percent by weight of C₂₋₄ hydrocarbons, preferably at least about 50 percent by weight.

The first step of the integrated process comprises catalytic production of benzene from a mixed lower alkane rich feedstock during which substantially all of C₃₊ hydrocarbons are converted in a single pass in this first step. In one embodiment, at least about 90% by weight of propane and heavier hydrocarbons in the feedstock is converted to aromatic hydrocarbons and byproducts, preferably at least about 95% by weight and most preferably at least about 99% by weight. The reaction may take place in the presence of a catalyst composition suitable for promoting the reaction of lower alkanes to aromatic hydrocarbons such as benzene. The reaction conditions may comprise a temperature of about 550 to about 750° C. and a pressure of about 0.01 to about 0.5 Mpa absolute.

Following a product separation scheme to recover all the aromatics and methane/hydrogen, the remaining C₂ rich stream is sent to the ethane cracking step, which may be a conventional ethane cracker (preferably catalytic or thermal), to produce ethylene. In this manner, the alkane to benzene reactor functions as a means of removing essentially all C₃₊ hydrocarbons from the feedstock going to the ethane cracker thus simplifying its design considerably. The capital and operating cost of the ethane cracker complex is significantly reduced by eliminating the necessity of separating small quantities of propylene from the ethylene which would be the case if the feed to the cracker contained a significant amount of C₃₋ hydrocarbons. In addition, the alkane to benzene process also is a single pass process (no recycle of unconverted feed) resulting in further capital and operating cost reduction for the overall integrated processing scheme described.

Any one of a variety of catalysts may be used to promote the reaction of lower alkanes to aromatic hydrocarbons. One such catalyst is described in U.S. Pat. No. 4,899,006 which is herein incorporated by reference in its entirety. The catalyst composition described therein comprises an aluminosilicate having gallium deposited thereon and/or an aluminosilicate in which cations have been exchanged with gallium ions. The molar ratio of silica to alumina is at least 5:1.

Another catalyst which may be used in the process of the present invention is described in EP 0 244 162. This catalyst comprises the catalyst described in the preceding paragraph and a Group VIII metal selected from rhodium and platinum. The aluminosilicates are said to preferably be MFI or MEL type structures and may be ZSM-5, ZSM-8, ZSM-11, ZSM-12 or ZSM-35.

Other catalysts which may be used in the process of the present invention are described in U.S. Pat. No. 7,186,871 and U.S. Pat. No. 7,186,872, both of which are herein incorporated by reference in their entirety. The first of these patents describes a platinum containing ZSM-5 crystalline zeolite synthesized by preparing the zeolite containing the aluminum and silicon in the framework, depositing platinum on the zeolite and calcining the zeolite. The second patent describes such a catalyst which contains gallium in the framework and is essentially aluminum-free.

Additional catalysts which may be used in the process of the present invention include those described in U.S. Pat. No. 5,227,557, hereby incorporated by reference in its entirety. These catalysts contain an MFI zeolite plus at least one noble metal from the platinum family and at least one additional metal chosen from the group consisting of tin, germanium, lead, and indium.

One preferred catalyst for use in this invention is described in U.S. Provisional Application No. 61/029,481, filed Feb. 18, 2008 entitled “Process for the Conversion of Ethane to Aromatic Hydrocarbons.” This application is hereby incorporated by reference in its entirety. This application describes a catalyst comprising: (1) about 0.005 to about 0.1% wt (% by weight) platinum, based on the metal, preferably about 0.01 to about 0.05% wt, (2) an amount of an attenuating metal selected from the group consisting of tin, lead, and germanium, which is no more than 0.02% wt less than the amount of platinum, preferably not more than about 0.2% wt of the catalyst, based on the metal; (3) about 10 to about 99.9% wt of an aluminosilicate, preferably a zeolite, based on the aluminosilicate, preferably about 30 to about 99.9% wt, preferably selected from the group consisting of ZSM-5, ZSM-11, ZSM-12, ZSM-23, or ZSM-35, preferably converted to the H+ form, preferably having a SiO₂/Al₂O₃ molar ratio of from about 20:1 to about 80:1, and (4) a binder, preferably selected from silica, alumina and mixtures thereof.

Another preferred catalyst for use in this invention is described in U.S. Provisional Application No. 61/029,939, filed Feb. 20, 2008 entitled “Process for the Conversion of Ethane to Aromatic Hydrocarbons.” This application is hereby incorporated by reference in its entirety. The application describes a catalyst comprising: (1) about 0.005 to about 0.1% wt (% by weight) platinum, based on the metal, preferably about 0.01 to about 0.06% wt, most preferably about 0.01 to about 0.05% wt, (2) an amount of iron which is equal to or greater than the amount of the platinum but not more than about 0.50% wt of the catalyst, preferably not more than about 0.20% wt of the catalyst, most preferably not more than about 0.10% wt of the catalyst, based on the metal; (3) about 10 to about 99.9% wt of an aluminosilicate, preferably a zeolite, based on the aluminosilicate, preferably about 30 to about 99.9% wt, preferably selected from the group consisting of ZSM-5, ZSM-11, ZSM-12, ZSM-23, or ZSM-35, preferably converted to the H+ form, preferably having a SiO₂/Al₂O₃ molar ratio of from about 20:1 to about 80:1, and (4) a binder, preferably selected from silica, alumina and mixtures thereof.

Another preferred catalyst for use in this invention is described in U.S. Provisional Application No. 61/029,478, filed Feb. 18, 2008 entitled “Process for the Conversion of Ethane to Aromatic Hydrocarbons.” This application is hereby incorporated by reference in its entirety. This application describes a catalyst comprising: (1) about 0.005 to about 0.1 wt % (% by weight) platinum, based on the metal, preferably about 0.01 to about 0.05% wt, most preferably about 0.02 to about 0.05% wt, (2) an amount of gallium which is equal to or greater than the amount of the platinum, preferably no more than about 1 wt %, most preferably no more than about 0.5 wt %, based on the metal; (3) about 10 to about 99.9 wt % of an aluminosilicate, preferably a zeolite, based on the aluminosilicate, preferably about 30 to about 99.9 wt %, preferably selected from the group consisting of ZSM-5, ZSM-11, ZSM-12, ZSM-23, or ZSM-35, preferably converted to the H+ form, preferably having a SiO₂/Al₂O₃ molar ratio of from about 20:1 to about 80:1, and (4) a binder, preferably selected from silica, alumina and mixtures thereof.

The hydrodealkylation reaction involves the reaction of toluene, xylenes, ethylbenzene, and higher aromatics with hydrogen to strip alkyl groups from the aromatic ring to produce additional benzene and light ends including methane and ethane which are separated from the benzene. This step substantially increases the overall yield of benzene and thus is highly advantageous.

Both thermal and catalytic hydrodealkylation processes are known in the art. Thermal dealkylation may be carried out as described in U.S. Pat. No. 4,806,700, which is herein incorporated by reference in its entirety. Hydrodealkylation operation temperatures in the described thermal process may range from about 500 to about 800° C. at the inlet to the hydrodealkylation reactor. The pressure may range from about 2000 kPa to about 7000 kPa. A liquid hourly space velocity in the range of about 0.5 to about 5.0 based upon available internal volume of the reaction vessel may be utilized. Due to the exothermic nature of the reaction, it is often required to perform the reaction in two or more stages with intermediate cooling or quenching of the reactants. Two or three or more reaction vessels may therefore be used in series. The cooling may be achieved by indirect heat exchange or interstage cooling. When two reaction vessels are employed in the hydrodealkylation zone, it is preferred that the first reaction vessel be essentially devoid of any internal structure and that the second vessel contain sufficient internal structure to promote plug flow of the reactants through a portion of the vessel.

Alternatively, the hydrodealkylation zone may contain a bed of a solid catalyst such as the catalyst described in U.S. Pat. No. 3,751,503, which is herein incorporated by reference in its entirety. Another possible catalytic hydrodealkylation process is described in U.S. Pat. No. 6,635,792, which is herein incorporated by reference in its entirety. This patent describes a hydrodealkylation process carried out over a zeolite-containing catalyst which also contains platinum and tin or lead. The process is preferentially performed at temperatures ranging from about 250° C. to about 600° C., pressures ranging from about 0.5 MPa to about 5.0 MPa, liquid hydrocarbon feed rates from about 0.5 to about 10 hr-1 weight hourly space velocity, and molar hydrogen/hydrocarbon feedstock ratios ranging from about 0.5 to about 10.

Lower Olefins, i.e. ethylene and propylene, may be produced from lower alkanes (ethane, propane and butane) by either thermal or catalytic cracking processes. The thermal cracking process may typically be carried out in the presence of superheated steam and this is by far the most common commercially practiced process. Steam cracking is a thermal cracking process in which saturated hydrocarbons (i.e. ethane, propane, butane or their mixture) are broken down into smaller, unsaturated hydrocarbons, i.e, olefins and hydrogen.

In steam cracking, the gaseous feed may be diluted with steam and then briefly heated in a furnace (without the presence of oxygen). Typically, the reaction temperature may be very high—around 750 to 950° C.—but the reaction is only allowed to take place very briefly. In modern cracking furnaces, the residence time may even be reduced to milliseconds (resulting in gas velocities reaching speeds beyond the speed of sound) in order to improve the yield of desired products. After the cracking temperature has been reached, the gas may quickly be quenched to stop the reaction in a transfer line heat exchanger.

The products produced in the reaction depend on the composition of the feed, the hydrocarbon to steam ratio and on the cracking temperature and furnace residence time. The process may typically be operated at low pressures, around 140 to 500 kPa depending on the overall process design.

The process may also result in the slow deposition of coke, a form of carbon, on the reactor walls. This degrades the efficiency of the reactor so reaction conditions are designed to minimize this. Nonetheless, a steam cracking furnace can usually only run for a few months at a time between de-cokings. De-cokings require the furnace to be isolated from the process and then a flow of steam or a steam/air mixture is passed through the furnace coils at high temperature. This converts the hard solid carbon layer to carbon monoxide and carbon dioxide. Once this reaction is complete, the furnace can be returned to service.

In many commercial operations, ethylene and propylene are separated from the resulting complex mixture by repeated compression and distillation at low temperatures. In the process of the present invention, this may be unnecessary because the feed to the cracker is mostly comprised of ethane.

The first stages of olefin production and purification in a cracker complex are: 1) steam cracking in furnaces as described above; 2) primary and secondary heat recovery with quench; 3) dilution steam recycle between the furnaces and the quench system; 4) primary compression of the cracked gas (multiple stages of compression); 5) hydrogen sulfide and carbon dioxide removal (acid gas removal); 6) secondary compression (1 or 2 stages); 7) drying of the cracked gas; and 8) cryogenic treatment of the dried, cracked gas.

The cold, cracked gas stream is then treated in a demethanizer. The overhead stream from the demethanizer, consisting of hydrogen and methane, is treated cryogenically to separate the hydrogen and methane. This separation step usually involves liquid methane at a temperature of about −150° C. Complete recovery of all the methane is critical to the economical operation of the olefin plant.

The bottom stream from the demethanizer tower is treated in a deethanizer tower. The overhead stream from the deethanizer tower consists of all the C₂, 's that were in the cracked gas stream. The C₂'s then go to a C₂ splitter. The product ethylene is taken from the overhead of the tower and the ethane coming from the bottom of the splitter is recycled to the furnaces to be cracked again.

The bottom stream from the deethanizer tower may go to a depropanizer tower but this may be eliminated in the process of this invention. The overhead stream from the depropanizer tower consists of all the C₃'s that were in the cracked gas stream. Prior to sending the C₃'s to the C₃ splitter this stream is hydrogenated in order to react out the methylacetylene and propadiene. Then this stream is sent to the C₃ splitter. The overhead stream from the C₃ splitter is product propylene and the bottom stream from the C₃ splitter is propane which can be sent back to the furnaces for cracking or used as fuel.

The bottom stream from the depropanizer tower may go to a debutanizer tower but this may also be eliminated in the process of this invention. The overhead stream from the debutanizer is all of the C₄'s that are in the cracked gas stream. The bottom stream from the debutanizer consists of everything in the cracked gas stream that is C₅ or heavier. This could be called a light pyrolysis gasoline.

Since the production of ethylene is energy intensive, much effort has been dedicated recovering heat from the gas leaving the furnaces. Most of the energy recovered from the cracked gas may be used to make high pressure (around 8300 kPa) steam. This steam may in turn be used to drive the turbines for compressing cracked gas, the propylene refrigeration compressor which may be unnecessary in the process of this invention, and the ethylene refrigeration compressor.

The ethylene manufacturing process may also accomplished by in the presence of a catalyst. The advantages are the use of much lower temperatures and possibly the absence of steam. In principle, a higher selectivity to olefins and possibly lower coke make can be achieved. Though it has not been practiced commercially at a world scale plant, catalytic cracking of ethane has been an area of interest for a long time. The types of catalysts used to crack higher hydrocarbons include zeolites, clays, aluminosilicates, and others. It should be mentioned that this process is practiced commercially in several oil refineries for high molecular weight hydrocarbons which are cracked over zeolite catalysts in a process unit called FCC (Fluidized Catalytic Cracker). It is more common in such processes to produce and recover propylene as a byproduct rather than both ethylene and propylene.

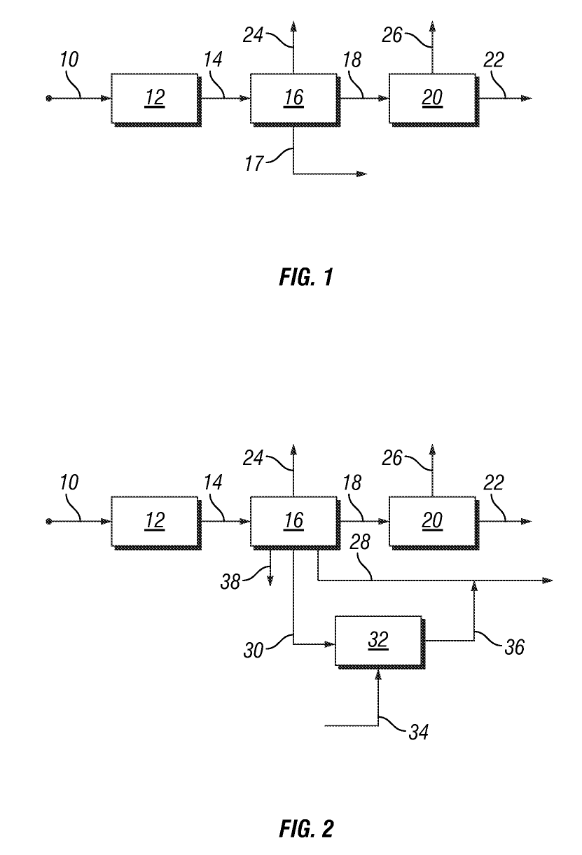

One embodiment of the concept of this invention is illustrated in the simplified block flow diagram in FIG. 1. In FIG. 1, the ethane/propane/butane-rich stream 10 is fed to a reactor 12 for converting alkanes to benzene containing a suitable catalyst or catalyst mixture. The reactor product stream 14 contains unreacted ethane and diluent (if any), plus hydrogen, methane, small amounts of C₃-C₅ hydrocarbons, benzene, toluene, xylenes and heavier aromatics, with selectivity to benzene preferably greater than about 20%. This product stream 14 passes through appropriate separation and extraction equipment 16 and the unreacted ethane 18 is fed to the ethane cracker 20 where it is converted to ethylene 22. The H₂ may be recovered optionally (but not necessarily) from the C₁ (methane) stream 24 from separation unit 16 and/or the similar stream 26 from cracker 20 using pressure swing adsorption or a membrane process and may be sent to a hydrodealkylation unit as described below. The aromatics leave separation unit 16 through line 17.

There are several variations to the process whose main objective is to produce ethylene and aromatics from a single mixed feedstock 10 containing ethane and higher hydrocarbons. In one version as shown in FIG. 1 of the aromatics, only the produced benzene is recovered. There is no hydrodealkylation unit and the toluene and xylenes co-produced are recovered along with the C₉₊ aromatics. In another version, as shown in FIG. 2, both toluene and xylenes are selectively converted into benzene and methane. This additional benzene is then added to the benzene produced in the main reaction. In another variation (not shown), no attempt is made to separate the benzene, toluene, and xylene components and their mixture is sent to the hydrodealkylation unit.

In FIG. 2, benzene is also separated from toluene and xylene in separation unit 16. The benzene leaves through line 28 and the toluene and xylene leave through line 30 and are directed to the hydrodealkylation unit 32 and combined with hydrogen from line 34. The toluene and xylene are hydrodealkylated to produce benzene in line 36 which may then be combined with benzene line 28. Additionally, C₉₊ aromatics are removed from separation unit 16 through line 38.

### EXAMPLES

The examples provided below are intended to illustrate but not limit the scope of the invention.

### Example 1

Catalysts A and B were made with low levels of Pt and Ga on extrudate samples containing 80% wt of CBV 2314 ZSM-5 powder (23:1 molar SiO₂:Al₂O₃ ratio, available from Zeolyst International) and 20% wt alumina binder. These catalysts were prepared as described in U.S. Provisional Application No. 61/029,478, filed Feb. 18, 2008 entitled “Process for the Conversion of Ethane to Aromatic Hydrocarbons.” The extrudate samples were calcined in air up to 650° C. to remove residual moisture prior to use in catalyst preparation. The target metal loadings for catalyst A were 0.025% w Pt and 0.09% wt Ga. The target metal loadings for catalyst B were 0.025% wt Pt and 0.15% wt Ga.

Metals were deposited on 25-50 gram samples of the above ZSM-5/alumina extrudate by first combining appropriate amounts of stock aqueous solutions of tetraammine platinum nitrate and gallium(III) nitrate, diluting this mixture with deionized water to a volume just sufficient to fill the pores of the extrudate, and impregnating the extrudate with this solution at room temperature and atmospheric pressure. Impregnated samples were aged at room temperature for 2-3 hours and then dried overnight at 100° C.

Catalysts made on the ZSM-5/alumina extrudate were tested “as is,” without crushing. For each performance test, a 15-cc charge of fresh (not previously tested) catalyst was loaded into a Type 316H stainless steel tube (1.40 cm i.d.) and positioned in a four-zone furnace connected to a gas flow system.

Prior to performance testing, the catalyst charges were pretreated in situ at atmospheric pressure (ca. 0.1 MPa absolute) as follows:
(a) calcination with air at 60 liters per hour (L/hr), during which the reactor wall temperature was increased from 25 to 510° C. in 12 hrs, held at 510° C. for 4-8 hrs, then further increased from 510 to 630° C. in 1 hr, then held at 630° C. for 30 min;
(b) nitrogen purge at 60 L/hr, 630° C. for 20 min;
(c) reduction with hydrogen at 60 L/hr, for 30 min, during which time the reactor wall temperature was raised from 630° C. to the temperature used for the actual run.

At the end of the above reduction step, the hydrogen flow was terminated, and the catalyst charge was exposed to a feed consisting of 67.2% wt ethane and 32.8% wt propane at atmospheric pressure (ca. 0.1 MPa absolute), 650-700° C. reactor wall temperature, and a feed rate of 500-1000 GHSV (500-1000 cc feed per cc catalyst per hr). Three minutes after introduction of the feed, the total reactor outlet stream was sampled by an online gas chromatograph for analysis. Based on composition data obtained from the gas chromatographic analysis, initial ethane, propane and total conversions were computed according to the following formulas:

ethane conversion, %=100×(% wt ethane in feed−% wt ethane in outlet stream)/(% wt ethane in feed)

propane conversion, %=100×(% wt propane in feed−% wt propane in outlet stream)/(% wt propane in feed)

total ethane+propane conversion=((% wt ethane in feed×% ethane conversion)+(% wt propane in feed×% propane conversion))/100

Table 1 lists the results of online gas chromatographic analyses of samples of the total product streams of these reactors taken at 3 minutes after introduction of the feed. Under these conditions, over 99% wt of the propane in the feed and over 55% w of the ethane in the feed was converted in all of these catalyst performance tests. The product stream contains benzene and higher aromatics, along with hydrogen and light hydrocarbons, including some ethane which can be recycled.

TABLE 1

| Catalyst | A | B | B | A | B | A |
|---|---|---|---|---|---|---|
| Catalyst charge weight, g | 11.58 | 11.52 | 12.36 | 11.43 | 11.51 | 11.73 |
| Reactor Wall Temperature, ° C. | 650 | 675 | 675 | 700 | 700 | 700 |
| Total feed rate, GHSV | 500 | 600 | 1000 | 800 | 800 | 1000 |
| Total feed rate, WHSV | 0.89 | 1.07 | 1.67 | 1.44 | 1.43 | 1.76 |
| % Ethane Conversion | 56.38 | 71.07 | 58.22 | 77.16 | 77.05 | 65.77 |
| % Propane Conversion | 99.3 | 99.48 | 99.11 | 99.61 | 99.61 | 99.5 |
| Total % (Ethane + Propane) Conversion | 70.51 | 80.4 | 71.64 | 84.53 | 84.45 | 76.84 |
| Reactor Outlet Composition, % wt | | | | | | |
| Hydrogen | 5.31 | 6.29 | 5.71 | 6.48 | 6.54 | 5.99 |
| Methane | 17.91 | 19.28 | 16.36 | 20.47 | 20.25 | 17.13 |
| Ethylene | 2.11 | 3.83 | 2.89 | 5.76 | 5.45 | 6.65 |
| Ethane | 29.26 | 19.43 | 28.06 | 15.34 | 15.42 | 22.99 |
| Propylene | 0.22 | 0.33 | 0.32 | 0.46 | 0.43 | 0.67 |
| Propane | 0.23 | 0.17 | 0.29 | 0.13 | 0.13 | 0.16 |
| C4 | 0.02 | 0.02 | 0.03 | 0.05 | 0.05 | 0.09 |
| C5 | 0 | 0 | 0 | 0 | 0 | 0 |
| Benzene | 26.97 | 29.68 | 27.45 | 30.06 | 30.34 | 24.99 |
| Toluene | 8.15 | 8.28 | 8.21 | 7.97 | 7.92 | 8.09 |
| C8 Aromatics | 0.74 | 0.83 | 0.79 | 0.94 | 0.88 | 1.06 |
| C9+ Aromatics | 9.06 | 11.86 | 9.88 | 12.33 | 12.58 | 12.17 |
| Total Aromatics | 44.93 | 50.84 | 46.33 | 51.31 | 51.73 | 46.31 |

### Example 2

Using fresh (not previously tested) charges of catalysts A and B described in Example 1 additional performance tests were conducted as described in Example 1 except that the feed consisted of 32.8% w ethane and 67.2% w propane. Table 2 lists the results of online gas chromatographic analyses of samples of the total product streams of these reactors taken at 3 minutes after introduction of the feed. Under these conditions, over 99% wt of the propane in the feed and over 20% w of the ethane in the feed was converted in all of these catalyst performance tests. The product stream contains benzene and higher aromatics, along with hydrogen and light hydrocarbons, including some ethane which can be recycled.

TABLE 2

| Catalyst | A | B | B | B | B | A |
|---|---|---|---|---|---|---|
| Catalyst charge weight, g | 11.58 | 11.51 | 11.52 | 11.93 | 12.36 | 11.73 |
| Reactor Wall Temperature, ° C. | 650 | 675 | 675 | 675 | 675 | 700 |
| Total feed rate, GHSV | 500 | 500 | 600 | 800 | 1000 | 800 |
| Total feed rate, WHSV | 0.99 | 0.98 | 1.22 | 1.57 | 1.9 | 1.6 |
| % Ethane Conversion | 23.73 | 59.12 | 48.81 | 42.53 | 36.17 | 66.32 |
| % Propane Conversion | 99.65 | 99.84 | 99.78 | 99.74 | 99.68 | 99.85 |
| Total % (Ethane + Propane) Conversion | 74.55 | 86.38 | 83.09 | 81 | 78.88 | 88.86 |
| Reactor Outlet Composition, % wt | | | | | | |
| Hydrogen | 4.79 | 5.7 | 5.45 | 5.61 | 5.78 | 5.78 |
| Methane | 19.65 | 23.7 | 22.34 | 19.64 | 17.05 | 24.54 |
| Ethylene | 2.88 | 2.95 | 3.1 | 3.67 | 4.17 | 4.44 |
| Ethane | 25.21 | 13.51 | 16.77 | 18.83 | 20.91 | 11.03 |
| Propylene | 0.27 | 0.21 | 0.27 | 0.38 | 0.45 | 0.37 |
| Propane | 0.23 | 0.11 | 0.14 | 0.17 | 0.21 | 0.1 |
| C4 | 0.03 | 0.01 | 0.02 | 0.05 | 0.06 | 0.04 |
| C5 | 0 | 0 | 0 | 0 | 0 | 0 |
| Benzene | 27.23 | 31.73 | 30.71 | 29.14 | 26.99 | 29.44 |
| Toluene | 9.28 | 7.75 | 8.77 | 9.82 | 10.17 | 7.69 |
| C8 Aromatics | 1.08 | 0.71 | 0.9 | 1.19 | 1.4 | 0.91 |
| C9+ Aromatics | 9.34 | 13.61 | 11.52 | 11.51 | 12.82 | 15.66 |
| Total Aromatics | 46.93 | 53.8 | 51.9 | 51.65 | 51.37 | 53.69 |

What is claimed is:

1. An integrated process for producing ethylene and aromatic hydrocarbons which comprises:
(a) contacting a mixed lower alkane feed with an aromatic hydrocarbon conversion catalyst to produce a product mixture which is comprised of aromatic reaction products including benzene, unreacted ethane and non-aromatic products,
(b) separating and recovering the benzene and any other aromatic reaction products,
(c) separating and recovering the ethane, and
(d) introducing the ethane into an alkane cracker to produce ethylene.

2. The process of claim 1 wherein the majority of the lower alkanes in the mixed lower alkane feed is comprised of ethane and propane.

3. The process of claim 1 wherein the mixed lower alkane feed is comprised of at least 30 percent by weight of C₂₋₄ hydrocarbons.

4. The process of claim 1 wherein the mixed lower alkane feed is comprised of at least 50 percent by weight of C₂₋₄ hydrocarbons.

5. An integrated process for producing ethylene and aromatic hydrocarbons which comprises:
(a) contacting a mixed lower alkane feed with an aromatic hydrocarbon conversion catalyst to produce a product mixture which is comprised of aromatic reaction products including benzene and toluene and/or xylene, unreacted ethane, and non-aromatic products,
(b) separating and recovering the aromatic reaction products,
(c) separating benzene from the other aromatic reaction products,
(d) hydrodealkylating the toluene and/or xylene to produce additional benzene,
(e) separating and recovering the ethane, and
(f) introducing the ethane into an alkane cracker to produce ethylene.

6. The process of claim 5 wherein the majority of the lower alkanes in the mixed lower alkane feed is comprised of ethane and propane.

7. The process of claim 5 wherein the mixed lower alkane feed is comprised of at least 30 percent by weight of C₂₋₄ hydrocarbons.

8. The process of claim 5 wherein the mixed lower alkane feed is comprised of at least 50 percent by weight of C₂₋₄ hydrocarbons.
